(19) United States
(12) Patent Application Publication
(10) Pub. No.: US 2011/0257361 A1
(43) Pub. Date: Oct. 20, 2011

(54) ALKYL BENZENE TETRACARBOXYLIC DIANHYDRIDE, MANUFACTURING METHOD THEREOF, POLYIMIDE, AND APPLICATION THEREOF

(76) Inventors: Hideo Suzuki, Chiba (JP); Takayuki Tamura, Chiba (JP); Takahiro Noda, Chiba (JP)

(21) Appl. No.: 13/140,880

(22) PCT Filed: Dec. 17, 2009
(86) PCT No.: PCT/JP2009/071026

§ 371 Date: Jun. 20, 2011

(30) Foreign Application Priority Data
Dec. 19, 2008 (JP) 2008-323587

(51) Int. Cl. C08G 73/10; C07C 51/377; C07C 63/313; C07D 493/04

(52) U.S. Cl. 528/353; 549/239; 562/483

(57) ABSTRACT

Disclosed is a 5-alkyl-1,2,3,4-benzene tetracarboxylic-1:2,3:4-dianhydride which is represented by the general formula (1) and has excellent solubility with respect to various organic solvents. Depending on the diamine that is used, a polyimide with excellent heat resistance or a polyimide with good workability at a low melting point can be provided, and in addition, a polyimide that exhibits excellent characteristics for electronic materials or the like can be provided.

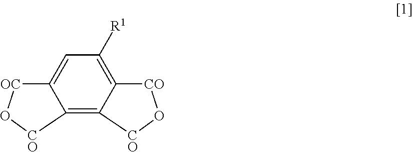

(In the formula, R¹ represents an alkyl group with carbon number 1-10.)

### TECHNICAL FIELD

This invention relates to an alkylbenzene tetracarboxylic dianhydride, its producing method, polyimides and a use thereof. More particularly, the invention relates, for example, to a polyimide suited for use as an electronic material and also to an alkylbenzene tetracarboxylic dianhydride that is a starting monomer thereof.

### BACKGROUND ART

Aromatic polyimide resins show excellent mechanical, thermal and chemical properties since they have a rigid molecular structure as taking a conjugated structure wherein an aromatic ring and an aromatic ring are directly bound through an imide bond having strong intermolecular forces.

Commercially sold polyimide resins not only have drastically higher strength and heat resistance than ordinary polymers, but also are excellent in electric insulation and enables wiring of high accuracy because of their linear coefficient of expansion that is very low as an organic matter and is close to metals. Thus, they have been hitherto employed as an insulating material of electronic circuits and the like.

In recent years, while making use of such characteristics of high electric insulation and solvent resistance, the resins have been widely used as a protecting or insulating material for liquid crystal display devices and semiconductors and also as an electronic material such as of color filters.

While aromatic polyimides have such advantages as mentioned above, a disadvantage is involved in their insolubility and infusibility because of the rigid molecular structure and strong intermolecular forces. Accordingly, the application thereof needs, after molding of a precursor, conversion into a polyimide.

For instance, a polyimide obtained by use of pyromellitic dianhydride as a tetracarboxylic dianhydride (Kapton (registered trade name) or the like), which is a typical example of hitherto known polyimides, has such properties that it is insoluble in organic solvents and is infusible by itself and a difficulty is involved in molding processability and limitation is placed on its use situation.

It has been reported that a polyimide making use, as a tetracarboxylic dianhydride, of an unsubstituted 1,2,3,4-benzenetetracarboxylic-1,2:3,4-dianhydride is soluble in amide organic solvents such as N-methyl-2-pyrrolidone and the like (Non-patent Document 1).

However, the amide organic solvents are all high in boiling point, leaving a problem on their separation by removal and thus, there is a demand for aromatic polyimides that are soluble in organic solvents having low boiling points.

### PRIOR ART DOCUMENT

### Non-Patent Document

Non-patent Document 1: 3Pb136 of the 56^{th} Symposium of the Society of Polymer Science, Japan

### SUMMARY OF THE INVENTION

### Problems to be Solved by the Invention

The invention has been made under such circumstances and has for its object of the provision of a tetracarboxylic dianhydride, which is excellent in solubility in various types of organic solvents and which is able to yield a polyimide that is excellent in heat resistance although depending on the type of diamine used or a polyimide having a low melting point and a good processability, a method for producing same, and a polyimide and an application thereof.

### Means for Solving the Problems

We made intensive studies so as to achieve the above object and, as a result, found that when using a starting monomer wherein an alkyl group is introduced into 1,2,3,4-benzenetetracarboxylic acid-1:2,3:4-dianhydride at the 5^{th} position thereof, there can be obtained a polyimide that is excellent in solubility in various types of organic solvents and is excellent in heat resistance although depending on the type of diamine used, or a polyimide having a low melting point and good processability and that these polyimides are capable of showing excellent characteristics when applied, for example, to liquid crystal display devices, thereby arriving at completion of the invention.

More particularly, the invention provides:

1. A 5-alkyl-1,2,3,4-benzenetetracarboxylic-1:2,3:4-dianhydride represented by the formula [1]

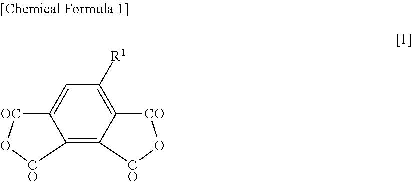

(wherein R¹ represents an alkyl group having 1 to 10 carbon atoms);
2. The 5-alkyl-1,2,3,4-benzenetetracarboxylic-1:2,3:4-dianhydride of 1, wherein R¹ is an n-butyl group;
3. A 5-alkyl-1,2,3,4-benzenetetracarboxylic acid represented by the formula [2]

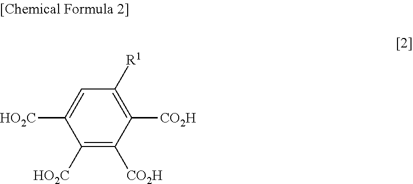

(wherein R¹ represents an alkyl group having 1 to 10 carbon atoms);
4. The 5-alkyl-1,2,3,4-benzenetetracarboxylic acid of 3, wherein R¹ is an n-butyl group;
5. A method for producing a 5-alkyl-1,2,3,4-benzenetetra-carboxylic-1:2,3:4-dianhydride represented by the formula [1]

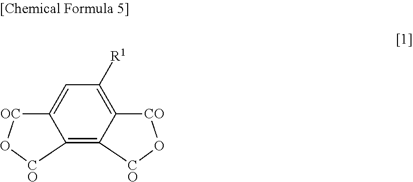

(wherein R¹ has the meaning as defined below), characterized by comprising hydrolyzing a tetraalkyl 5-alkyl-1,2,3,4-benzenetetracarboxylate represented by the formula [3]

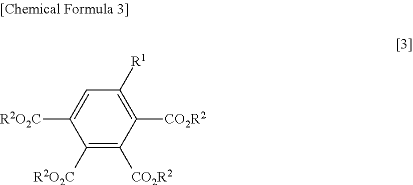

(wherein R¹ and R² independently represent an alkyl group having 1 to 10 carbon atoms) to obtain a 5-alkyl-1,2,3,4-benzenetetracarboxylic acid represented by the formula (2)

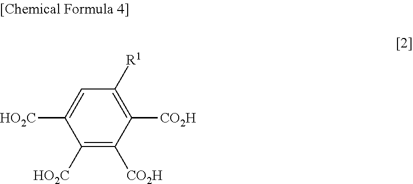

(wherein R¹ has the same meaning as defined above), and subsequently subjecting to dehydration and ring closure;
6. A method for producing a 5-alkyl-1,2,3,4-benzenetetra-carboxylic acid represented by the formula (2)

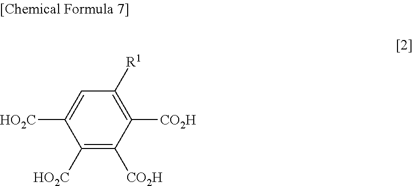

(wherein R¹ has the same meaning as defined below), characterized by comprising hydrolyzing a tetraalkyl t-alkyl-1,2,3,4-benzenetetracarboxylate represented by the formula [3]

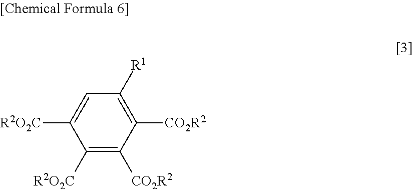

(wherein R¹ and R² independently represent an alkyl group having 1 to 10 carbon atoms).
7. A polyamic acid containing at least 10 mol % of recurring units represented by the formula [6]

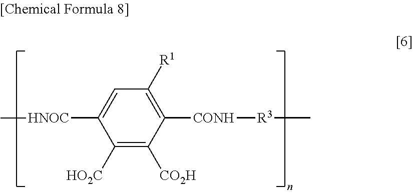

(wherein R¹ represents an alkyl group having 1 to 10 carbon atoms, R³ represents a divalent organic group, and n is an integer of 2 or over);
8. The polyamic acid of 7, wherein R¹ is an n-butyl group;
9. A polyimide containing at least 10 mole % of recurring units represented by the formula [7]

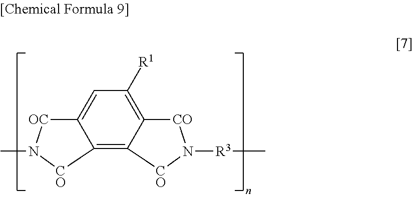

(wherein R¹ represents an alkyl group having 1 to 10 carbon atoms, R³ represents a divalent organic group, and n is an integer of 2 or over);
10. The polyimide of 9, wherein R¹ is an n-butyl group;
11. A liquid crystal orientation processor, characterized by comprising the polyamic acid or polyimide of any one of 7 to 10.
12. A crystal liquid orientation film obtained from the liquid crystal orientation processor of 11.
13. A liquid crystal display device provided with the liquid crystal orientation film of 12.

### ADVANTAGEOUS EFFECT OF THE INVENTION

According to the invention, there can be provided an aromatic tetracarboxylic dianhydride, which is a starting monomer capable of yielding a polyimide that is excellent in solubility in various types of organic solvents and is also excellent in heat resistance although depending on the type of diamine used, or a polyimide has low melting point and good processability.

The polyimide of the invention obtained from this aromatic tetracarboxylic dianhydride used as a starting material can be conveniently used, for example, as a protecting material for liquid crystal display devices or semiconductors, as an electronic material such as an insulating material or as a material for optical communication such as an optical waveguide.

### BRIEF DESCRIPTION OF THE DRAWINGS

FIG. 1 is a ¹H-NMR spectrum of BBDA-DDE polyimide obtained in Example 6.

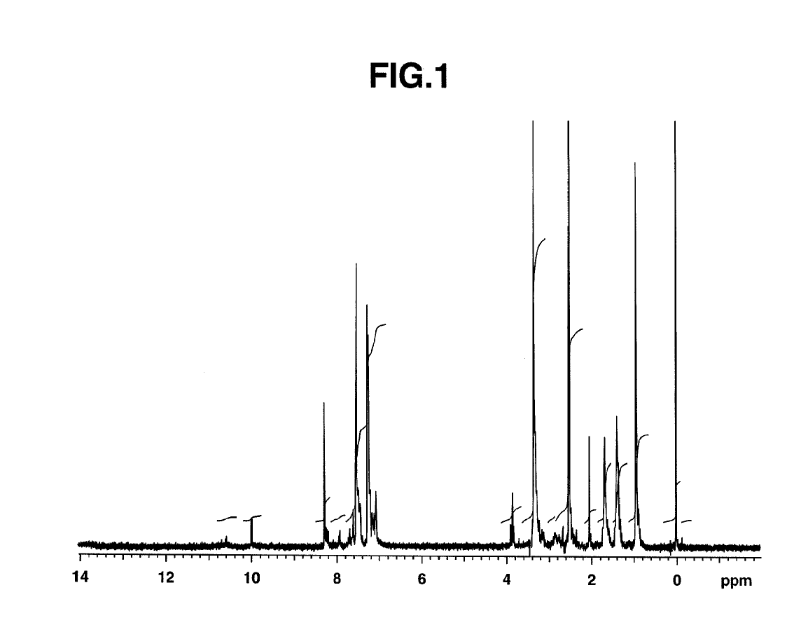

FIG. 2 is a ¹H-NMR spectrum of BBDA-DA4P polyimide obtained in Example 7.

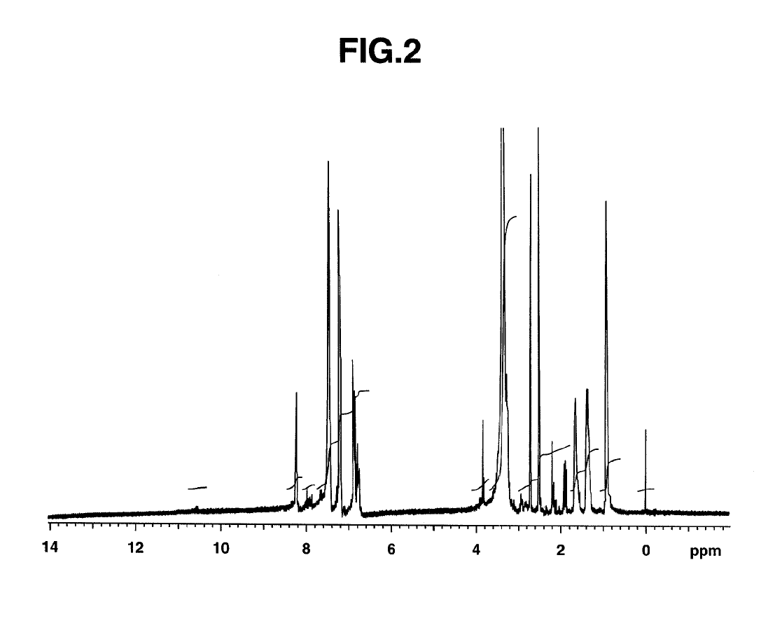

FIG. 3 is a ¹H-NMR spectrum of BBDA-DA5MG polyimide obtained in Example 8.

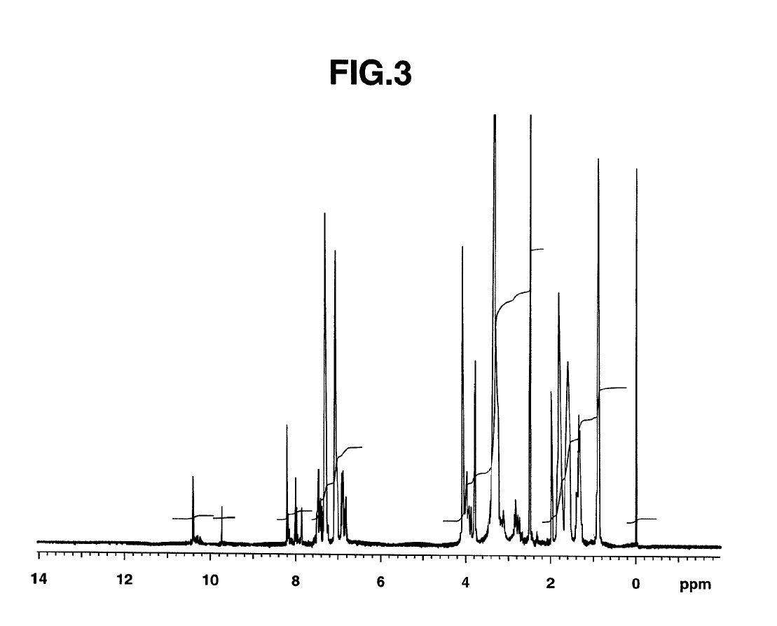

FIG. 4 is a ¹H-NMR spectrum of BBDA-PDA polyimide obtained in Example 9.

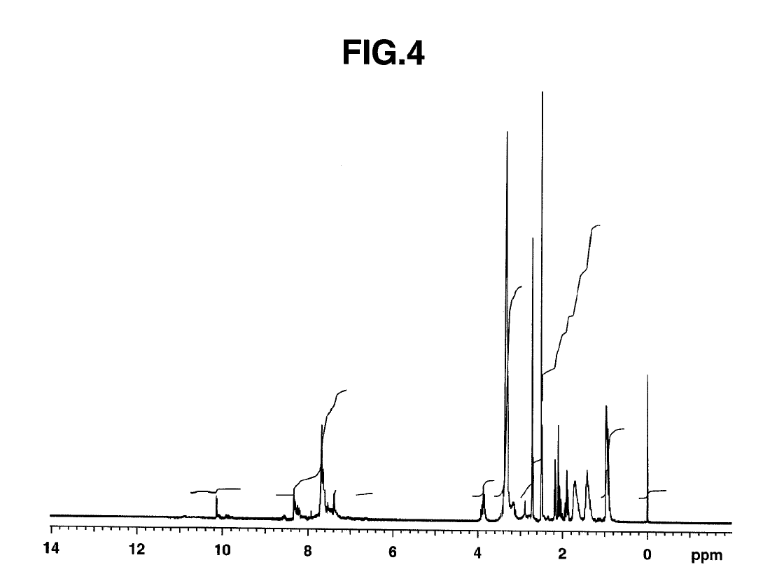

### EMBODIMENT FOR CARRYING OUT THE INVENTION

The invention is now described in more detail.

It will be noted that indicated by n is normal, by i is iso, by s is secondary, by t is tertiary, and by c is cyclo, respectively, hereinafter.

In the above formulas, the alkyl group having 1 to 10 carbon atoms may be any of linear, branched and cyclic ones and specific examples include methyl, ethyl, n-propyl, i-propyl, c-propyl, n-butyl, i-butyl, s-butyl, t-butyl, c-butyl, n-pentyl, 1-methyl-n-butyl, 2-methyl-n-butyl, 3-methyl-n-butyl, 1,1-dimethyl-n-propyl, c-pentyl, 2-methyl-c-butyl, n-hexyl, 1-methyl-n-pentyl, 2-methyl-n-pentyl, 1,1-dimethyl-n-butyl, 1-ethyl-n-butyl, 1,1,2-trimethyl-n-propyl, c-hexyl, 1-methyl-c-pentyl, 1-ethyl-c-butyl, 1,2-dimethyl-c-butyl, n-heptyl, n-octyl, n-nonyl, n-decyl and the like.

Among them, when taking into account enhanced solubility of the resultant polyimides in organic solvents, there is preferably used as R¹ an alkyl group having 2 to 10 carbon atoms, more preferably an alkyl group having 3 to 10 carbon atoms, and much more preferably an alkyl group having 4 to 10 carbon atoms.

The method for producing a 5-alkyl-1,2,3,4-benzene-tetracarboxylic dianhydride (hereinafter abbreviated as ABDA) represented by the formula [1] can be expressed according to a series of reaction schemes indicated below.

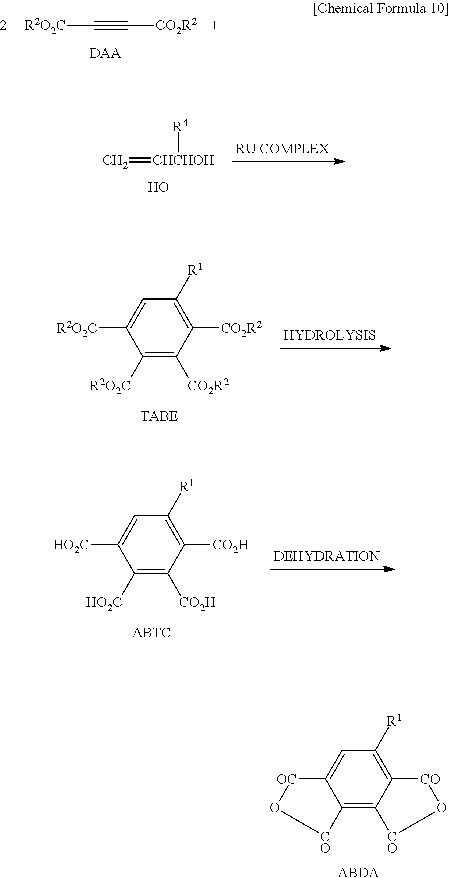

(wherein R¹ and R², respectively, have the same meanings as defined above, and R⁴ represents a hydrogen atom or an alkyl group having 1 to 9 carbon atoms).

More particularly, the first step is a step of preparing a tetraalkyl 5-alkyl-1,2,3,4-benzenetetracarboxylate (hereinafter abbreviated as TABE) from a dialkylacetylenedicarboxylate (DAA) and a (substituted) allylic alcohol (HO) by use of a ruthenium complex as a catalyst. This step can be carried out according the procedure set out in JP-A 2001-19662.

No specific limitation is placed on the dialkylacetylene dicarboxylate so far as it has an alkyl group (R²) having 1 to 10 carbon atoms. Specific examples include dimethylacetylene dicarboxylate, diethylacetylene dicarboxylate, dipropyleneacetylene dicarboxylate, dibuthylacetylene dicarboxylate, dipentylacetylene dicarboxylate, dicyclopentylacetylene dicarboxylate, dihexylacetylene dicarboxylate, dicyclohexylacetylene dicarboxylate, diheptylacetylene dicarboxylate, dioctylacetylene dicarboxylate, dinonylacetylene dicarboxylate, didecylacetylene dicarboxylate and the like. Of these, dimethylacetylene dicarboxylate is preferred because of the ease in availability.

No specific limitation is also placed on the allylic alcohol so far as it is able to yield an alkyl group (R¹) having 1 to 10 carbon atoms. Specific examples include allyl alcohol, 3-buten-2-ol, 1-penten-3-ol, 1-hexen-3-ol, 1-hepten-3-ol, 1-octen-3-ol, 1-nonen-3-ol, 1-decen-3-ol, 1-undecen-3-ol, 1-dodecen-3-ol and the like.

The second step is one wherein TABE obtained in the first step is hydrolyzed to obtain a 5-alkyl-1,2,3,4-benzenetetracarboxylic acid (hereinafter abbreviated as ABTC).

In this case, although the hydrolysis technique may adopt ordinary conditions, which are carried out in the presence of a base or acid so as to obtain ordinary carboxylic acid compounds from ester compounds, it is preferred to carry out the hydrolysis in the presence of a base.

The base used may be a hydroxide of an alkali metal or an alkali earth metal. More particularly, sodium hydroxide or potassium hydroxide is preferred in view of economy. The amount is preferably 4 to 10 times by mol of TABE, more preferably 5 to 8 times by mole.

The solvent of the hydrolytic reaction is preferably a mixed one of water and an organic solvent. Examples of the organic solvent include alcohols such as methanol, ethanol and the like, and 1,4-dioxane and the like. The amount is preferably 1 to 10 times by weight, more preferably 2 to 8 times by weight, of TABA for each of water and an organic solvent.

The reaction temperature is at approximately 0 to 200° C., preferably at 0 to 150° C.

After the reaction, the solution is rendered acidic with hydrochloric acid water and extracted such as with ethyl acetate, followed by concentration thereby obtaining crude crystals of ABTC. In order to further increase purity, the crude crystals are re-dissolved in ethyl acetate while heating, to which n-heptane is added, followed by ice-cooling thereby permitting crystals of high purity to be precipitated.

The third step is one wherein ABTC obtained in the second step is dehydrated to obtain ABDA.

As the dehydration method, mention is made of (a) an aliphatic carboxylic anhydride method, (b) a formic acid and p-toluenesulfonic acid method, (c) an azeotropic method using an aromatic hydrocarbon, and like.

Of these, it is preferred in the practice of the invention to use (a) an aliphatic carboxylic anhydride method because of the operational simplicity and a higher yield of an intended product.

The aliphatic carboxylic anhydride includes, for example, acetic anhydride, propionic anhydride and the like, of which acetic anhydride is preferred in view of economy.

The amount of the aliphatic carboxylic anhydride is preferably 2 to 20 times by mol, more preferably 3 to 10 times by mol, of the starting ABTC.

The above dehydration reaction is preferably carried out in coexistence of an aromatic hydrocarbon compound. In this step, the reaction solution is colored as the reaction proceeds and the crystals of the product are apt to suffer coloration. If an aromatic hydrocarbon compound coexists, the coloration of the reaction solution can be reduced, with the result that the coloration of the resultant product can be suppressed.

The aromatic hydrocarbon compound is not specifically limited and mention is made of benzene, toluene, xylene, ethylbenzene, cumene and the like, of which toluene is preferred in view of economy.

The amount of the aromatic hydrocarbon compound is preferably 1 to 30 times by weight, more preferably 3 to 20 times by weight, of the starting ABTC.

The reaction temperature is generally at about 50 to 150° C. and is preferably at 80 to 130° C. when consideration is taken to the shortage of time before completion of the reaction.

If the reaction time is long, the coloration of the resulting solution is enhanced, so that the time is preferably at 15 minutes to 3 hours, more preferably at 30 minutes to 2 hours.

It will be noted that the reaction can be carried out in the presence of active carbon for the purpose of decoloration. In this case, the amount of active carbon is preferably at 1 to 30 wt %, more preferably at 3 to 20 wt %, relative to the starting ABTC.

The completion of the reaction can be judged in terms of complete dissolution of the starting ABTC after raising the temperature.

After the reaction, crystals precipitated after ice cooling under agitation are filtered and washed, and further dried to obtain target ABDA.

The reactions of the above-stated steps can be all carried out at a normal pressure or under pressure in a batchwise manner or continuously.

The alkylbenzene tetracarboxylic dianhydride of the invention set forth hereinabove can be converted to a corresponding polyimide by subjecting it to polycondensation with a diamine to obtain a polyamic acid, followed by dehydration and ring-closing reactions making use of heat or a catalyst.

The alkylbenzene tetracarboxylic dianhydride of the invention yields polyamides whose melting points differ depending on the type of diamine, and also yields a polyimide having an excellent heat resistance or a polyimide having a low melting point and good processability although depending on the type of diamine used.

The diamine is not specifically limited in type and various types of diamines employed in existing preparation of polyimides can be used. Specific examples include: aromatic diamines such as p-phenylenediamine, m-phenylenediamine, 2,5-diaminotoluene, 2,6-diaminotoluene, 4,4′-diaminobiphenyl, 3,3′-dimethyl-4,4′-diaminophenyl, 3,3′-dimethoxy-4,4′-diaminobiphenyl, diaminodiphenylmethane, diaminodiphenyl ether, 2,2′-diaminodipehnylpropane, bis(3,5-diethyl-4-aminophenyl)methane, diaminodiphenylsulfone, diaminobenzophenone, diaminonaphthalene, 1,4-bis(4-aminophenoxy)benzene, 1,4-bis(4-aminophenyl)benzene, bis(4-aminophenoxy)pentane, 9,10-bis(4-aminophenyl)anthracene, 1,3-bis(4-aminophenoxy)benzene, 3,5-diamino-1,6-dimethoxybenzene, 3,5-diamino-1,6-dimethoxytoluene, 4,4′-bis(4-aminophenoxy)diphenylsulfone, 2,2-bis[4-(4-aminophenoxy)phenyl]propane, 2,2′-trifluoromethyl-4,4′-diaminobiphenyl and the like; alicyclic diamines such as 4,4′-methylenebis(cyclohexylamine), 4,4′-methylenebis(2-methylcyclohexylamine), bis(4-aminocyclohexyl)ether, bis(4-amino-3-methylcyclohexyl)ether, bis(4-aminocyclohexyl)sulfide, bis(4-amino-3-methylcyclohexyl)sulfide, bis(4-aminocyclohexyl)sulfone, bis(4-amino-3-methylcyclohexyl)sulfone, 2,2-bis(4-amincyclohexyl)propane, 2,2-bis(4-amino-3-methylcyclohexyl)propane, bis(4-aminocclohexyl)dimethylsilane, bis(4-amino-3-methylcyclohexyl)dimethylsilane and the like; and aliphatic diamines such as tetramethylendiamine, hexamethylenediamine and the like. These diamines may be used singly or in admixture of two or more.

As a diamine that is incorporated as a diamine component for the preparation of a polyamic acid or polyimide so that the pre-tilt angle of a liquid crystal can be increased when formed as a liquid crystal orientation film, there are known diamines having a substituent such as a long-chain alkyl group, a perfluoroalkyl group, an aromatic cyclic group, an alicyclic group or a combination thereof, a steroid skeleton group or the like.

In the practice of the invention, these diamines can be used in combination with the acid dianhydride represented by the formula [1].

Specific examples of the diamines having such a substituent are indicated below although not limited thereto. It will be noted that in the structures exemplified below, j is an integer of 5 to 20 and k is an integer of 1 to 20.

It will also be noted that R³ in the above formulas [6] and [7] is a divalent organic group derived from an employed diamine.

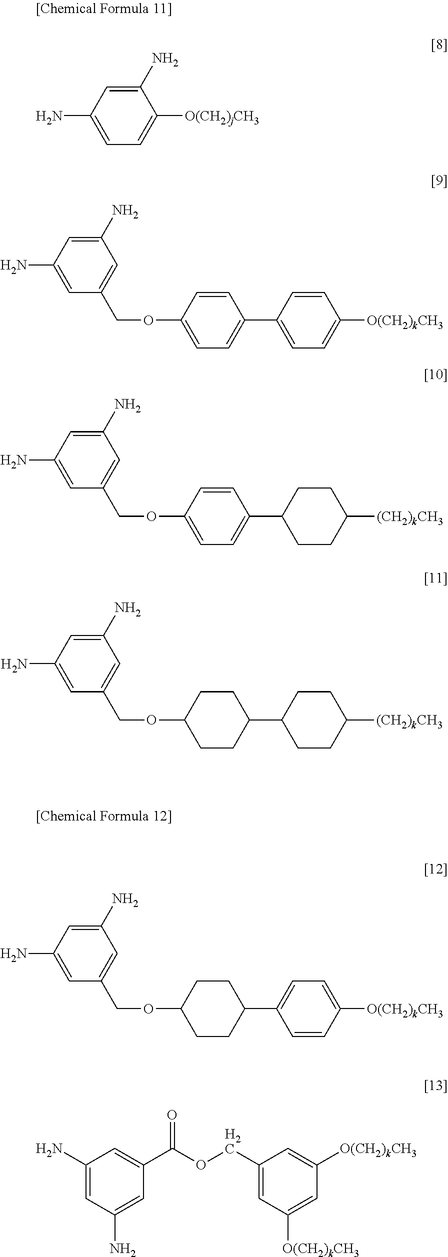

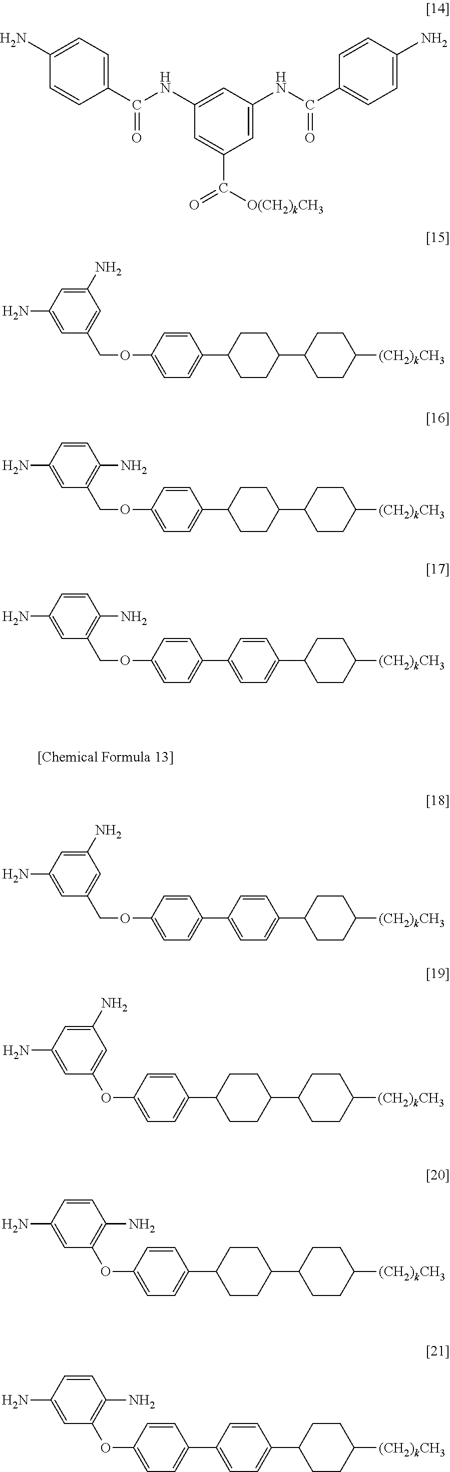

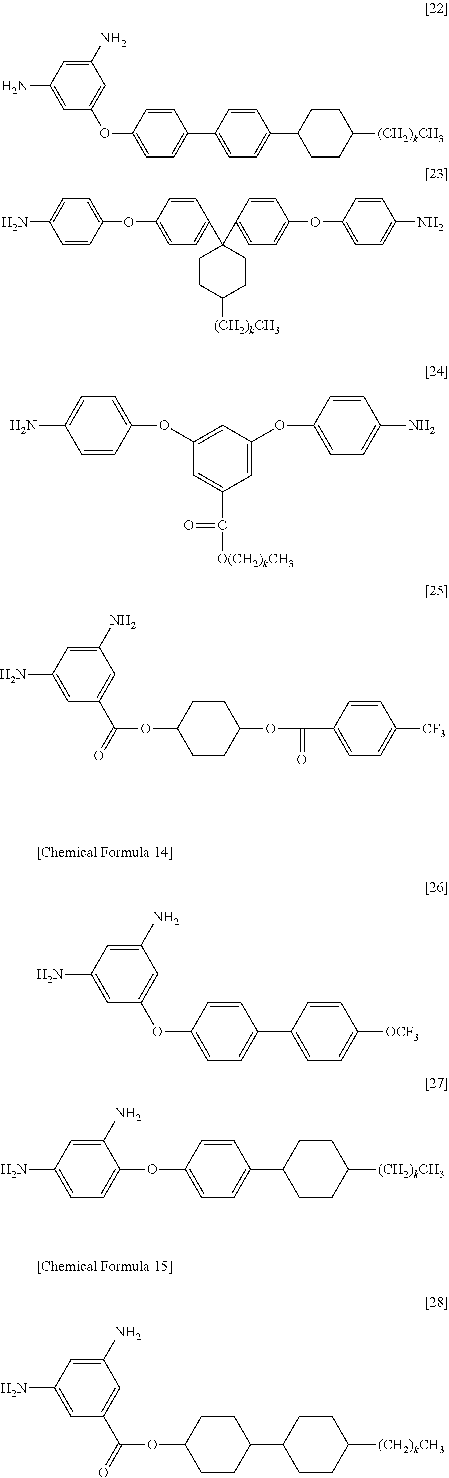

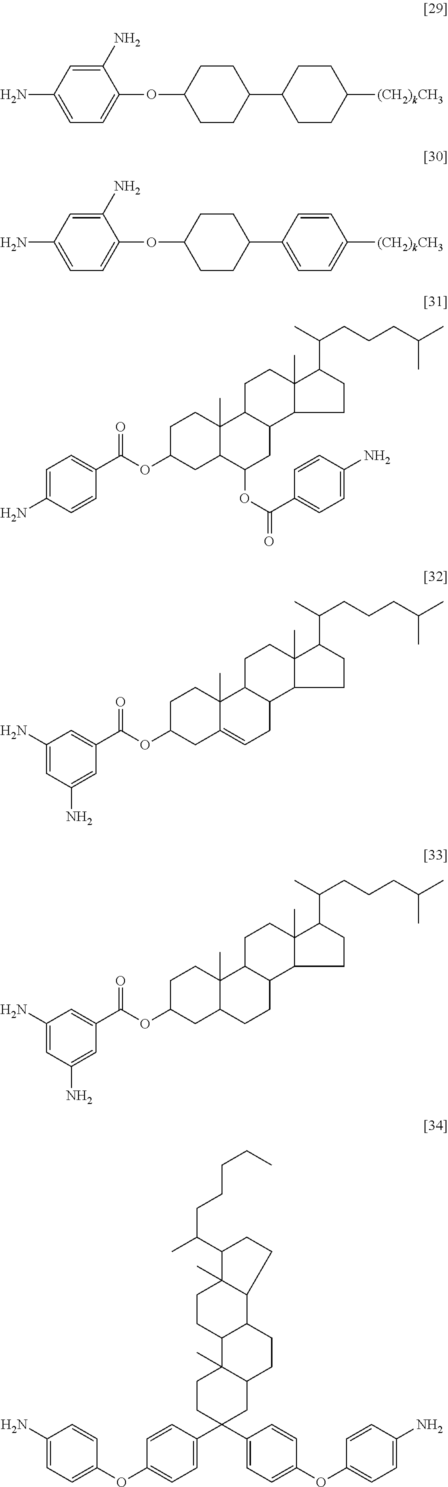

Among the above-indicated diamines, the diamine of the formula [8] is preferred because of its excellent liquid crystal orientation property. The diamines of the formulas [15] to [22] exhibit very high tilt-angle developability and can be conveniently used as an orientation film for OCB (optically compensated bend) liquid crystal (hereinafter referred to as orientation film for OCB) and an orientation film for vertical orientation mode liquid crystal (hereinafter referred to as orientation film for VA).

For instance, with an orientation film for TN liquid crystal (pre-tilt angle: 3 to 5°), the content of the diamine of the formula [8] is preferably set at 10 to 30 mol % relative to the total of diamine components, and with the orientation film for OCB or with the orientation film for VA (pre-tile angle: 10 to 90°), the content of the diamines of the formulas [15] to [22] is preferably set at 5 to 40 mol % relative to the total of diamine components although not limited thereto.

In the practice of the invention, at least 10 mol % of the total moles of tetracarboxylic acid dianhydrides used should be made up of ABDA of the formula [1]. Moreover, in order to achieve high solubility in organic solvent for the purpose of the invention, not less than 50 mol % of tetracarboxylic dianhydrides is preferably made up of ABDA, more preferably not less than 70 mol % is made up of ABDA, and optimally, not less than 90 mol % is made up of ABDA.

It is to be noted that in so far as ABDA is contained at not less than 10 mol %, a tetracarboxylic acid compound and a derivative thereof, which are used for ordinary polyamide preparation, may be simultaneously used.

Specific examples include alicyclic tetracarboxylic acids, acid dianhydrides thereof, such as
1,2,3,4-cyclobutanetetracarboxylic acid,
2,3,4,5-tetrandyrofuranetetracarboxylic acid,
1,2,4,5-cyclohexanoic acid,
3,4-dicarboxy-1-cyclohexylsuccinic acid,
3,4-dicarboxy-1,2,3,4-tetrahydro-1-naphthalenesuccinic acid,
bicyclo[3.3.0]octane-2,4,6,8-tetracarboxylic acid
   and the like, and dicarboxylic acid diacid halides thereof.

Additionally, mention may be made of aromatic tetracarboxylic acids, and acid dianhydrides thereof, such as pyromellitic acid, 2,3,6,7-naphthalenetetracarboxylic acid, 1,2,5,6-anthacenetetracarboxylic acid, 1,4,5,8-naphthalenetetracarboxylic acid, 2,3,6,7-anthracenetetracarboxylic acid, 1,2,5,6-anthracenetetracarboxylic acid, 3,3′,4,4′-biphenyltetracarboxylic acid, 2,3,3′,4-biphenyltetracarboxylic acid, bis(3,4-dicarboxyphenyl)ether, 3,3′,4,4′-benzophenonetetracarboxylic acid, bis(3,4-dicarboxyphenyl)methane, 2,2-bis(3,4-dicarboxyphenyl)propane, 1,1,1,3,3,3-hexafluoro-2,2-bis(3,4-dicarboxyphenyl)propane, bis(3,4-dicarboxyphenyl)dimethylsilane, bis(3,4-dicarboxyphenyl)diphenylsilane, 2,3,4,5-pyridinetetracarboxylic acid, 2,6-bis(3,4-dicarboxyphenyl)pyridine and the like, and dicarboxylic acid diacid halides thereof. It will be noted that these tetracarboxylic acid compounds may be used singly or in admixture of two or more.

The method of obtaining a polyamic acid according to the invention is not specifically limited and a tetracarboxylic dianhydride and a derivative thereof and a diamine may be reacted and polymerized by known techniques.

The ratio by mol of all tetracarboxylic dianhydride compounds and all diamine compounds used for preparation of a polyamic is preferably such that carboxylic acid compounds/diamine compounds=0.8 to 1.2. Like ordinary polycondensation reactions, the molar ratio closer to 1 leads to a greater degree of polymerization of the resulting polymer. If the degree of polymerization is too small, strength becomes unsatisfactory when the resulting polyimide is formed as a film. If the degree of polymerization is too large, there may be the case where workability in the course of the formation of a polyimide film becomes worsened.

Accordingly, the degree of polymerization of a product obtained in this reaction is preferably at 0.05 to 5.0 dl/g (at a concentration of 0.5 g/dl in N-methyl-2-pyrrolidone of 30° C.) in terms of reduced viscosity of a polyamic acid solution.

The solvents used for the preparation of polyamic acid include, for example, m-cresol, N-methyl-2-pyrrolidone (hereinafter abbreviated as NMP), N,N-dimethylformamide (hereinafter abbreviated as DMF), N,N-dimethylacetamide (hereinafter abbreviated as DMAc), N-methylcaprolactam, dimethylsulfoxide, tetramethylurea, pyridine, dimethylsulfone, hexamethylphosphoramide, γ-butyrolactone and the like. These may be used singly or in admixture thereof. Moreover, solvents incapable of dissolution of polyamic acids may be used, in addition to the above-indicated solvents, within a range where a uniform solution is obtainable.

The temperature of the polycondensation reaction is selected from arbitrary temperature of −20 to 150° C., preferably −5 to 100° C.

The polyimide of the invention can be obtained by dehydration and ring-closure (thermal imidization) of the thus prepared polyamic acid by heating. It will be noted that it is possible that a polyamic acid is converted to an imide in a solvent for use as a solvent-soluble polyimide.

Chemical ring closing methods making use of known dehydrating, ring closing catalysts may also be adopted.

The method based on the heating can be carried out at an arbitrary temperature of 100 to 350° C., preferably 120 to 300° C.

The chemical ring-closing method can be carried out, for example, in the presence such as with pyridine, triethylamine or the like and acetic anhydride or the like.

In this case, the temperature can be selected from arbitrary temperatures of −20 to 20° C.

The polyimide solution obtained in this way may be used as it is, may be used by admixing with a poor solvent such as methanol, ethanol or the like to permit the polyimide to be precipitated, followed by isolation as a polyimide powder, or may be used after re-dissolving the polyimide powder in an appropriate solvent.

The solvent for re-dissolution is not critical so far as it is able to dissolve the resulting polyimide. Examples include m-cresol, 2-pyrrolidone, NMP, N-ethyl-2-pyrrolidone, N-vinyl-2-pyrrolidone, DMAc, DMF, γ-butyrolactone and the like.

Such solvents incapable of dissolving polyimides when used singly may be used, in addition to the above-indicated solvents, within a range not impeding the solubility. Specific examples include ethyl cellosolve, butyl cellosolve, ethyl carbitol, butyl carbitol, ethyl carbitol acetate, ethylene glycol, 1-methoxy-2-propanol, 1-ethoxy-2-propanol, 1-butoxy-2-propanol, 1-phenoxy-2-propanol, propylene glycol monoacetate, propylene glycol diacetate, propylene glycol-1-monomethyl ether-2-acetate, propylene glycol-1-monoethyl ether-2-acetate, dipropylene glycol, 2-(2-ethoxypropoxy)propanol, lactic acid methyl ester, lactic acid ethyl ester, lactic acid n-propyl ester, lactic acid n-butyl ester, lactic acid isoamyl ester and the like.

In the practice of the invention, the number average molecular weight of the polyimide (polyamic acid) is preferably at least 5,000, more preferably 6,000 to 100,000 when taking into account flexibility when formed as a film.

Hence, n in the foregoing formulas, which is defined as an integer of 2 or over, is preferably an integer enough to realize a number average molecular weight of not smaller than 5,000 and particularly, is at 8 to 180, preferably at 10 to 100.

The thus prepared polyamic acid (polyimide precursor) solution is coated onto a substrate and subjected to dehydration and ring closure while evaporating the solvent by heating, or the polyimide solution is coated onto a substrate and heated to evaporate the solvent, thereby preparing a polyimide film.

The heating temperature is generally at about 100 to 300° C.

It will be noted that for the purpose of further improving adhesion between the polyimide film and the substrate, additives such as a coupling agent and the like may be added to a polyamic acid solution or a polyimide solution.

Examples of the additives, such as a coupling agent and the like, for improving film characteristics include silane coupling agents such as
3-aminopropylmethyldiethoxysilane,
3-phenylaminopropyltriemthoxysilane,
3-aminopropyltriethoxysilane,
   (aminoethylaminomethyl)phenethyltrimethoxysilane and the like. Although film adhesion to a substrate can be improved by the addition of these silane coupling agents, an excess amount causes a resin component such as a polyamic acid or polyimide to be coagulated. Preferably, the coupling agent relative to the resin component such as a polyamic acid or polyimide is at 0.5 to 10 wt %, more preferably 1 to 5 wt %.

The solid content of the liquid crystal orientation processor of the invention may be appropriately changed depending on the thickness of a liquid crystal orientation film to be formed and is preferably at 1 to 10 wt %. If the amount is smaller than 1 wt %, a difficulty is involved in forming a uniform, defect-free film. Over 10 wt %, solution storage stability may be worsened in some case. Although the solid content of a polyamic acid or polyimide of the invention is not critical, it is preferably at not smaller than 1 wt %, more preferably at not smaller than 3 wt % and much more preferably at not smaller than 5 wt %, from the standpoint of characteristics of the resulting liquid crystal orientation film.

The liquid crystal orientation processor obtained in this manner is preferably filtered prior to coating onto a substrate.

The liquid crystal orientation processor of the invention is coated onto a substrate, dried and baked to provide a film, and can be used as a liquid crystal orientation film for rubbing by subjecting the film surface to rubbing. The film may also be used as a liquid crystal orientation film for VA, not subjected to rubbing, or a photo-orientation film.

In this case, the substrate used is not critical so far as substrates of high transparency are used. To this end, there can be used plastic substrates such as an acrylic substrate, a polycarbonate substrate and the like. It is preferred from the standpoint of process simplification to use substrates forming an ITO electrode or the like thereon for liquid crystal driving. With a reflection type liquid crystal display device, an opaque material such as a silicon wafer or the like may be used only for a one-sided substrate. In this case, a material capable of reflecting light, such as aluminum or the like, may be used as an electrode.

As a method of coating a liquid crystal orientation processor, mention is made of a spin coating method, a printing method, an inkjet method and the like. In view of productivity, a flexo printing method has been industrially in wide use and is favorably used for the liquid crystal orientation processor of the invention.

The drying step after coating of a liquid crystal orientation processor is not always necessary. Nevertheless, if a time after coating to baking is not constant for every substrate or if baking is not effected immediately after coating, it is preferred to include the drying step. Drying is such that a solvent is evaporated to such as extent as not to allow film shape deformation such as by the transfer of substrate and drying means is not specifically limited. For a specific instance, mention is made of a method of drying on a hot plate at 50 to 150° C., preferably 80 to 120° C. for 0.5 to 30 minutes, preferably 1 to 5 minutes.

The substrate, on which the liquid crystal orientation processor has been coated, can be baked at an arbitrary temperature of 100 to 350° C., preferably 150 to 300° C. and more preferably 180 to 250° C. In case where an amic acid group exists in the liquid crystal orientation processor, a conversion rate of the amic acid to an imide varies depending on the baking temperature. In this connection, however, the liquid crystal orientation processor of the invention need not be always imidized to 100%.

As to the film thickness after baking, too large a thickness is disadvantageous from the standpoint of the consumed electric power of the liquid crystal display device and too small a thickness may lower the reliability of the liquid crystal display device. Thus, the thickness is preferably at 10 to 200 nm, more preferably at 50 to 100 nm.

The rubbing treatment of the film surface formed on the substrate in this way may be carried out by use of existing rubbing apparatus. The rubbing cloth material includes cotton, rayon, nylon and the like.

Using the thus obtained liquid crystal orientation film-attached substrate, a liquid crystal cell can be made according to a known technique to provide a liquid crystal display device. For an instance of making a liquid crystal cell, it is usual to use a method wherein a pair of substrates, on which a liquid crystal orientation film has been formed, sandwich therebetween a spacer whose thickness is preferably 1 to 30 μm, more preferably 2 to 10 μm and is so set that a rubbing direction is at an arbitrary angle of 0 to 270°, followed by fixing with a sealing agent therearound, injecting a liquid crystal and sealing. Although the method of sealingly injecting a liquid crystal is not critical, there may be exemplified a vacuum method wherein after pressure is reduced within the thus made liquid cell, a liquid crystal is injected, a dropping method wherein after a liquid crystal is dropped, sealing is made, and the like.

The thus obtained liquid crystal display device can be conveniently used for various types of display devices including TN liquid crystal display devices, STN liquid crystal display devices, TFT liquid crystal display devices and OCB liquid crystal display devices along further with lateral electric field type liquid crystal display devices, VA liquid crystal display devices and the like.

### EXAMPLES

The invention is more particularly described by way of Synthetic Examples, Examples and Comparative Examples and the invention should not be construed as limited to the following Examples. The measuring apparatuses of the respective physical properties in the Examples are those indicated below.

### [1] Mass Analysis (MASS)

Model: LX-1000 (JEOL Ltd.), detection method: FAB method

### [2] ¹H-NMR:

Model: INOVA500 (VARIAN Corp.), measuring solvent: DMSO-d₆

Reference substance: tetramethylsilane (TMS)

### [3] Melting Point (m.p.)

Model: Micro melting point apparatus (MP-S3) (made by Yanaco Co., Ltd.)

### [4] Measurement of Number Average Molecular Weight and Weight Average Molecular Weight

The weight average molecular weight (hereinafter abbreviated as Mw) and molecular weight distribution of polymer was measured by use of a GPC apparatus (Shodex (registered trade name) Column KF803L and KF805L), made by Jasco Corporation, under conditions of a flow rate of DMF, used as an elution solvent, of 1 ml/minute and a column temperature of 50° C. It will be noted that Mw was a value converted to polystyrene.

### Synthetic Example 1

### First Step

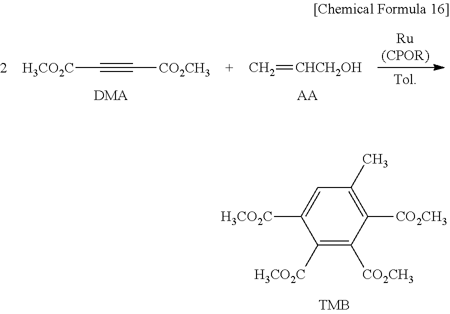

1.40 g (24 mmols) of allyl alcohol (AA), 26 g of toluene, 0.156 g (0.6 mmols) of triphenylphosphine and 0.228 g (0.6 mmols) of chloro(pentamethylcyclopentadienyl)-ruthenium(1,5-cyclooctadiene) complex [Cp*RuCl(cod)] were charged into a 100 ml four-necked reaction flask. The resulting reaction solution was heated to 70° C., in which 6.42 g (45 mmols) of dimethylacetylene dicarboxylate (DMA) was dropped in 20 minutes. The inner temperature was gently raised to 103° C. (bath temperature: 120° C.), followed by agitation for 2 hours.

After completion of the reaction, the solution was cooled and admixed with water and ethyl acetate, followed by liquid separation, washing the resulting organic phase and removing the solvent by distillation under reduced pressure to obtain 7.15 g of a crude oily matter. This crude matter was purified twice by silica gel column chromatography (eluant: ethyl acetate/heptane=1/3 to 1/0) to obtain 3.9 g (39.1 mmols, with an isolation yield of 53.4%) of crystals. It was confirmed from the results of MASS and ¹H-NMR analyses that this substance was tetramethyl 5-methyl-1,2,3,4-benzenetetracarboxylate (TMB).

### Synthetic Example 2

### First Step

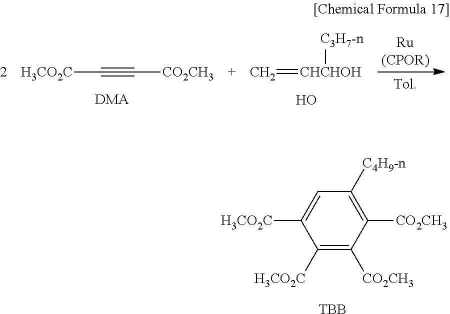

28.4 g (200 mmols) of dimethylacetylene dicarboxylate (DMA), 12.0 g (120 mmols) of 1-hexen-3-ol (HO), 284 g of toluene, 0.16 g (0.6 mmols) of triphenylphosphine and 1.01 g (2.66 mmols) of Cp*RuCl (cod) were charged into a 1000 ml four-necked reaction flask. When this reaction solution was heated to 90° C. in 30 minutes, the temperature was raised to 94° C. by the heat generation of the reaction. In a short time, the temperature went down to 90° C., for which the bath temperature was raised to 120° C., followed by agitation for 3 hours while refluxing at an inner temperature of 107° C.

After completion of the reaction, the solution was cooled and allowed to stand, whereupon a black solid matter was attached to the reaction flask. The solution was taken out by decantation and washed with water three times, after which the solvent was distilled off under reduced pressure to obtain 33.1 g of a crude oily matter. This crude matter was purified twice by silica gel column chromatography (eluant:ethyl acetate/heptane=1/3 to 1/1) to obtain 17.1 g of a purified product (46.6 mmols, with an isolation yield of 46.6%). It was confirmed from the results of MASS and ¹H-NMR analyses that this substance was tetramethyl 5-n-butyl-1,2,3,4-benzenetetracarboxylate (TBB).

### Example 1

### Second Step

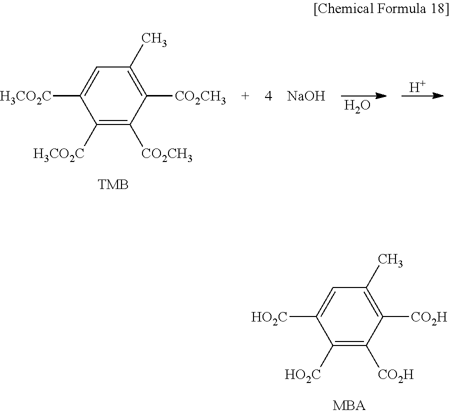

6.49 g (20 mmols) of TMS and 33 g of methanol were charged into a 100 ml four-necked reaction flask, to which a solution of 4.8 g (120 mmols) of sodium hydroxide dissolved in 20 g of water was added. The mixture solution was refluxed on a hot water bath for 8 hours. After completion of reaction, water was added to after concentration, which was subsequently rendered acidic by means of 35% hydrochloric acid. After further concentration, dioxane was added to the resulting residue and heated, followed by filtration and concentration of the resulting filtrate to obtain 4.72 g of crystals.

The crystals were re-crystallized from ethyl acetate and n-heptane to obtain 3.81 g (14.2 mmols, with an isolation yield of 71.0%) of white crystals.

It was confirmed from the results of MASS, ¹H-NMR and ¹³C-NMR analyses that this substance was 5-methyl-1,2,3,4-benzenetetracarboxylic acid (MBA).

MASS (ESI⁺, m/z (%)): 269 ([M+H]⁺, 13), 251 (100), 233 (98)

¹H-NMR (DMSO-d₆, δ ppm): 2.3783 (s, 3H), 7.7873 (s, 1H), 13.4827 (s, 4H)

¹³C-NMR (DMSO-d₆, δ ppm): 19.5957, 131.5146, 131.5833, 132.9034, 132.9721, 136.6119, 137.1384, 167.3940, 168.1113, 168.6073, 168.9964

m.p.: 199 to 200° C.

### Example 2

### Second Step

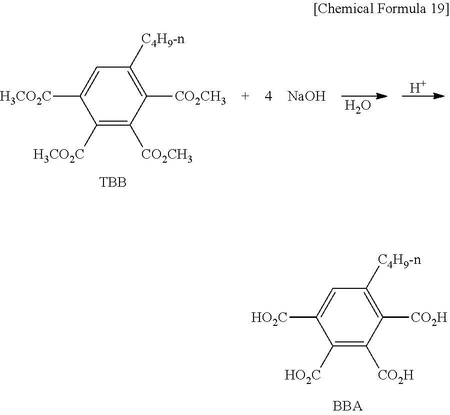

11.0 g (30 mmols) of TBB and 33 g of methanol were charged into a 100 ml four-necked reaction flask, to which a solution of 7.2 g (180 mmols) of sodium hydroxide dissolved in 33 g of water was added. The mixture solution was refluxed on a hot water bath for 8 hours. After completion of reaction, water was added to after concentration, which was subsequently rendered acidic by means of 35% hydrochloric acid. After further concentration, ethyl acetate and water were added to the resulting residue and heated, followed by concentration of the resulting organic phase to obtain crude crystals. On the other hand, after concentration of the aqueous phase, acetonitrile was added thereto and heated, followed by filtration, addition of the crude crystals to the resulting filtrate, and concentration to obtain 9.2 g of crystals.

The crystals were re-crystallized from ethyl acetate and n-heptane to obtain 7.33 g (23.6 mmols, with an isolation yield of 78.7%) of white crystals.

From the results of MASS, ¹H-NMR and ¹³C-NMR analyses, it was confirmed that this substance was 5-n-butyl-1,2,3,4-benzenetetracarboxylic acid (BBA).

MASS (ESI⁺, m/z (%)): 311 ([M+H]⁺, 11), 293 (93), 275 (100)

¹H-NMR (DMSO-d₆, δ ppm): 0.8813 (t, J=7.35 Hz, 3H), 1.2618 to 1.3357 (m, 2H), 1.5110 to 1.5416 (m, 2H), 2.7081 (t, J=7.65 Hz, 2H), 8.0731 (s, 1H), 12.9959 (brs, 4H)

¹³C-NMR (DMSO-d₆, δ ppm):14.2009, 22.4649, 32.7510, 33.3233, 131.5452, 131.7207, 132.3158, 132.9568, 136.8790, 141.2132, 167.4245, 168.1571, 168.6225, 169.0651

m.p.: 206 to 207° C.

### Example 3

### Second Step

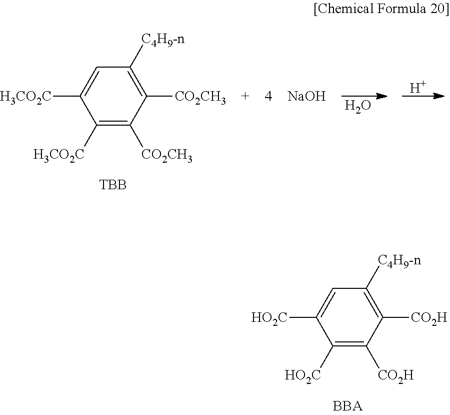

13.1 g (35.8 mmols) of TBB and 41 g of methanol were charged into a 100 ml four-necked reaction flask, to which a solution of 8.7 g (215 mmols) of sodium hydroxide dissolved in 41 g of water was added. The mixture solution was refluxed on a hot water bath of 90° C. for 8 hours. After completion of reaction, water was added to after concentration, which was subsequently rendered acidic by means of 35% hydrochloric acid. After further concentration, ethyl acetate and water were added to the resulting residue and heated, followed by concentration of the resulting organic phase to obtain 10.9 g (isolation yield: 92%) of flesh-colored crystals.

From the results of MASS, ¹H-NMR and ¹³C-NMR analyses, it was confirmed that this substance was 5-n-butyl-1,2,3,4-benzenetetracarboxylic acid (BBA).

### Example 4

### Third Step

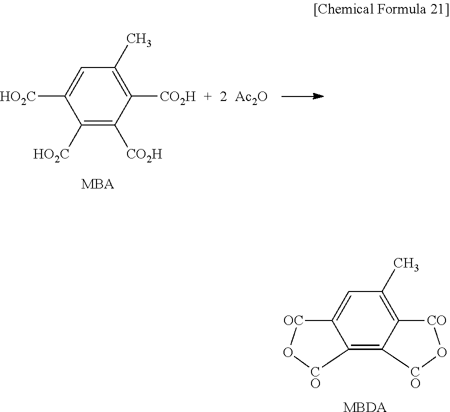

3.2 g (14.5 mmols) of MBA, 11.5 g (113 mmols) of acetic anhydride and 11.5 g of toluene were charged into a 100 ml four-necked flask, and this mixture solution was agitated under reflux on a hot water bath of 130° C. (inner temperature 108° C.) for 1 hour. Subsequently, ethyl acetate was added to a concentrated residue for dissolution by heating, after which n-heptane was added, followed by concentration to an extent that crystals commenced to precipitate, ice-cooling and filtration. The cake obtained by the filtration was washed with a mixture of ethyl acetate/n-heptane=1/1 and dried under reduced pressure to obtain 1.42 g (5.2 mmols, with an isolation yield of 35.7%) of light brown crystals.

Next, ethyl acetate was added to the crystals and heated for dissolution, and ice-cooled, whereupon crystals precipitated. After filtration and washing, drying under reduced pressure permitted 0.62 g (2.3 mmols, with an isolation yield of 15.6%) of light yellow crystals to be obtained.

From the results of MASS, ¹H-NMR and ¹³C-NMR analyses, it was confirmed that this substance was 5-methyl-1,2,3,4-benzenetetracarboxylic acid-1,2:3,4-dianhydride (MBDA).

MASS (ESI⁺, m/z): 233 ([M+H]⁺, 100)

¹H-NMR (500 MHz, DMSO-d₆, δ ppm): 2.82 (s, 3H), 8.49 (s, 1H)

¹³C-NMR (500 MHz, DMSO-d₆, δ ppm): 18.05, 125.77, 128.49, 132.92, 135.31, 137.78, 147.17, 158.63, 161.78

### Example 5

### Third Step

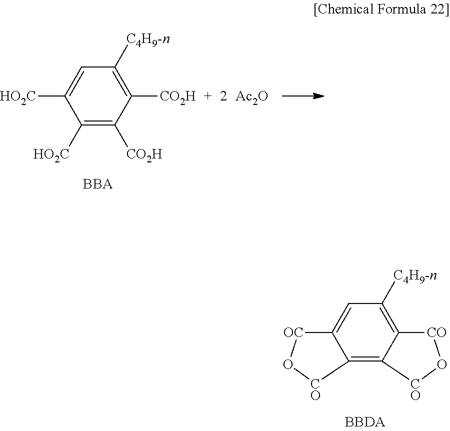

6.8 g (21.9 mmols) of BBA, 17.9 g (175 mmols) of acetic anhydride and 36 g of toluene were charged into a 100 ml four-necked flask, and this mixture solution was agitated under reflux on a hot water bath of 130° C. (inner temperature 108° C.) for 15 minutes to provide a uniform, light yellow solution. 1.36 g of active carbon was added to the solution, which was again agitated under reflux on a hot water bath of 130° C. (inner temperature 108° C.) for 30 minutes, followed by thermal filtration and concentration of the resulting filtrate to obtain 6.1 g of an oily matter. This oily matter solidified at 25° C., to which 30 ml of ethyl acetate was added, followed by dissolution on a hot water bath of 80° C. and subsequent addition of n-heptane, whereupon crystals precipitated.

Further, after ice-cooling, filtration was made, followed by washing with n-heptane and drying under reduced pressure to obtain 5.13 g (18.7 mmols, with an isolation yield of 85.4%) of white crystals.

From the results of MASS and ¹H-NMR analyses, it was confirmed that this substance was 5-n-butyl-1,2,3,4-benzenetetracarboxylic acid-1,2:3,4-dianhydride (BBDA).

MASS (ES⁺, m/z): 275 ([M+H]⁺, 100)

¹H-NMR (300 MHz, DMSO-d₆, δ ppm): 0.872 to 0.938 (m, 3H), 1.320 to 1.412 (m, 2H), 1.593 to 1.691 (m, 2H), 3.21 (t, J=4.0 Hz, 2H) 8.49 (s, 1H)

m.p.: 101 to 102° C.

### Example 6

### Synthesis of BBDA-DDE Polyamide Acid and Polyimide

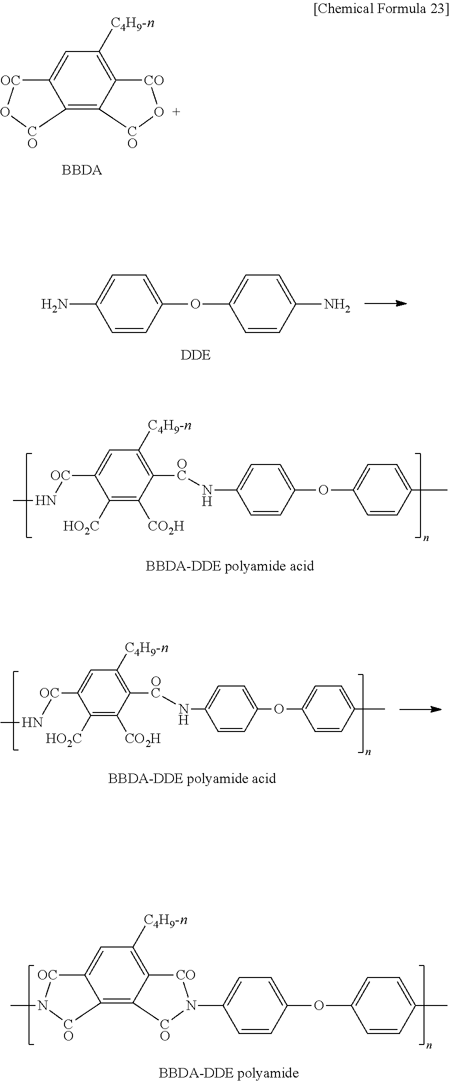

0.601 g (3.0 mmols) of 4,4′-diaminodiphenyl ether (hereinafter abbreviated as DDE) and 7.3 g of NPM were charged into a 50 ml four-necked reaction flask set in a water bath of 25° C. and equipped with an agitator, and dissolved. Subsequently, while agitating the resulting solution, 0.864 g (3.15 mmols) of BBDA was added in fractions to the solution under dissolution. Thereafter, the polymerization reaction was carried out under agitation at 22° C. for 26 hours to obtain a polyamide acid solution having a solid content of 20 wt %.

17 g of NMP, 6.12 g (60 mmols) of acetic anhydride and 2.85 g (36 mmols) of pyridine were added to the solution and agitated at 45° C. for 6 hours. This solution was cooled to room temperature and dropped in 3.5 times by volume of methanol, followed by further agitation for 1 hour to have a yellow powder precipitated. After filtration of the yellow powder, washing with methanol was repeated, followed by drying under reduced pressure at 80° C. for 3 hours to obtain 1.18 g (yield: 90%) of a yellow powder of BBDA-DDE polyimide.

This powder was subjected to measurement of the molecular weight by the GPC method (Gel Permeation Chromatography), with the result that the number average molecular weight (Mn) was 8,037 and the weight average molecular weight (Mw) was 14,871, with Mw/Mn being at 1.85.

m.p.: >300° C.

### Example 7

### Synthesis of BBDA-DA4P Polyamide Acid and Polyimide

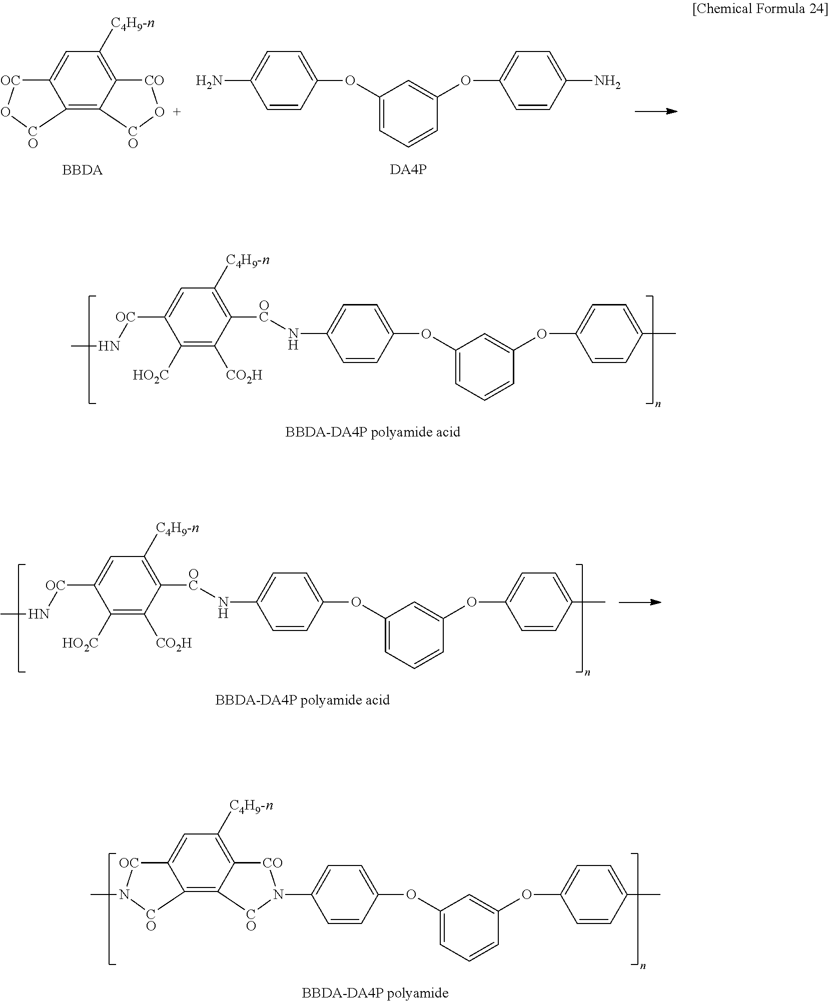

0.838 g (3.0 mmols) of 1,3-bis(4,4′-aminophenoxy)benzene (hereinafter abbreviated as DA4P) and 8.71 g of NMP were charged into a 50 ml four-necked flask set in a water bath of 25° C. and equipped with an agitator, and dissolved. Subsequently, while agitating the resulting solution, 0.905 g (3.3 mmols) of BBDA was added thereto in fractions under dissolution. Further, the polymerization reaction was carried out under agitation at 21° C. for 25 hours to obtain a polyamide acid solution having a solid content of 17 wt %.

27 g of NMP, 6.12 g (60 mmols) of acetic anhydride and 2.85 g (36 mmols) of pyridine were added to the solution and agitated at 45° C. for 6 hours. After cooling to room temperature, the reaction solution was dropped in 3.5 times by volume of methanol, followed by further agitation for 1 hour to have a yellow powder precipitated. After filtration of the yellow powder, washing with methanol was repeated, followed by drying under reduced pressure at 80° C. for 3 hours to obtain 1.47 g (yield: 954) of a yellow powder of BBDA-DA4P polyimide.

This powder was subjected to GPC measurement, with the result that the number average molecular weight (Mn) was 6,489 and the weight average molecular weight (Mw) was 10,629, with Mw/Mn being at 1.64.

m.p.: 225 to 230° C.

### Example 8

### Synthesis of BBDA-DA5MG polyamide Acid and Polyimide

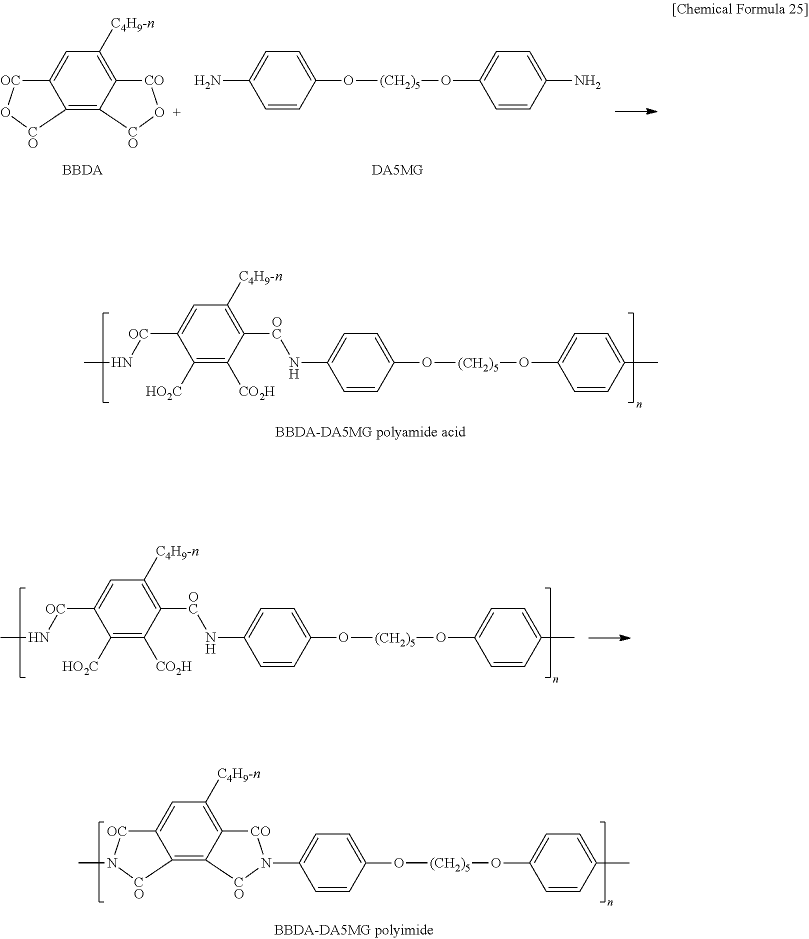

0.859 g (3.0 mmols) of 4,4′-diamino-1,5-phenoxypentane (hereinafter abbreviated as DA5MG) and 7.1 g of NMP were charged into a 50 ml four-necked flask set in a water bath of 25° C. and equipped with an agitator, and dissolved. Subsequently, while agitating the resulting solution, 0.905 g (3.3 mmols) of BBDA were added thereto in fractions under dissolution. Further, the polymerization reaction was carried out under agitation at 20° C. for 24 hours to obtain a polyamide acid solution having a solid content of 20 wt %.

22 g of NMP, 6.12 g (60 mmols) of acetic anhydride and 2.85 g (36 mmols) of pyridine were added to the solution and agitated at 45° C. for 6 hours and 30 minutes. The reaction solution was dropped in 3.5 times by volume of methanol and further agitated for 1 hour, followed by allowing the resulting precipitated yellow powder slurry to stand, whereupon a gum-like matter precipitated. This gum-like matter was dissolved in 20 g of DMF and was again dropped in methanol and re-precipitated, followed by filtration, washing three times the resulting filtrate with methanol and drying under reduced pressure at 80° C. for 3 hours to obtain 0.92 g (yield: 59%) of a yellow powder of BBDA-DA5MG polyimide.

This powder was subjected to GPC measurement, with the result that the number average molecular weight (Mn) was 4,227 and the weight average molecular weight (Mw) was 5,844, with Mw/Mn being at 1.38.

m.p.: 160 to 165° C.

### Example 9

### Synthesis of BBDA-PDA Polyamide Acid and Polyimide

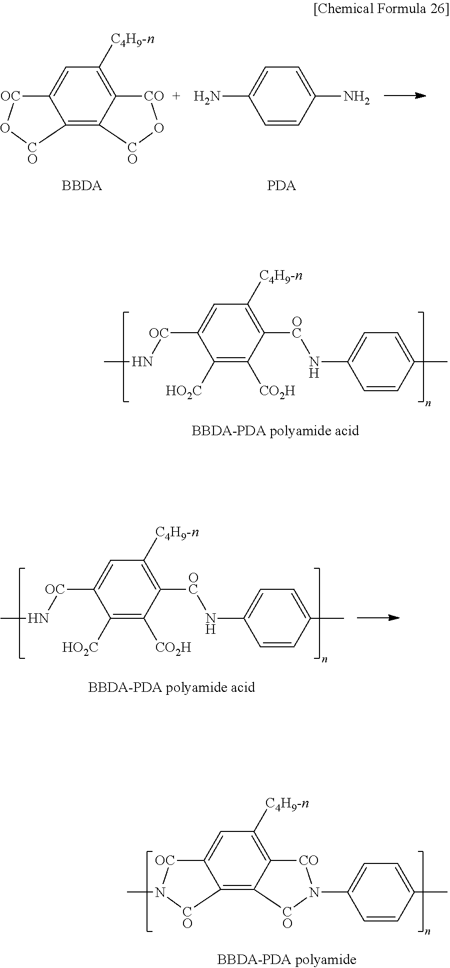

0.433 g (4.0 mmols) of p-phenylenediamine (hereinafter abbreviated as PDA) and 8.22 g of NMP were charged into a 50 ml four-necked flask set in a water bath of 25° C. and equipped with an agitator, and dissolved. Subsequently, while agitating the resulting solution, 1.22 g (4.4 mmols) of BBDA were added thereto in fractions under dissolution. Further, the polymerization reaction was carried out under agitation at 20° C. for 24 hours to obtain a polyamide acid solution having a solid content of 20 wt %.

22 g of NMP, 8.22 g (80 mmols) of acetic anhydride and 3.80 g (40 mmols) of pyridine were added to the solution and agitated at 45° C. for 5 hours and 30 minutes. The reaction solution was dropped in 3.5 times by volume of methanol, followed by further agitation for 1 hour to have an orange powder precipitated. The orange powder was filtered, followed by washing with water three times and drying under reduced pressure at 80° C. for 3 hours to obtain 1.37 g (yield: 99%) of an orange powder of BBDA-PDA polyimide.

This powder was subjected to GPC measurement, with the result that the number average molecular weight (Mn) was 2,460 and the weight average molecular weight (Mw) was 3,572, with Mw/Mn being at 1.45.

m.p.: 275 to 280° C.

### Comparative Example 1

### Synthesis of PMDA-DDE Polyamide Acid and Polyimide

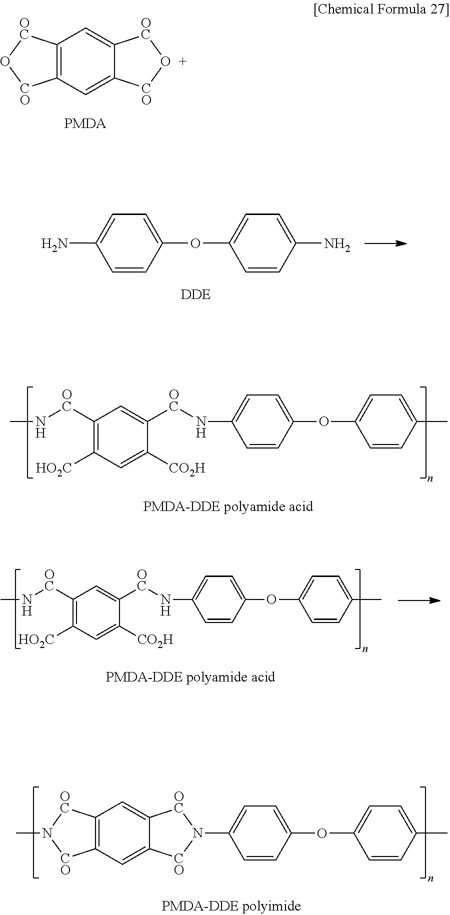

1.00 g (5.0 mmols) of DDE and 18.8 g of NMP were charged into a 50 ml four-necked flask set in a water bath of 25° C. and equipped with an agitator, and dissolved. Subsequently, while agitating the resulting solution, 1.09 g (5 mmols) of pyromellitic dianhydride (PMDA) were added thereto in fractions under dissolution. Further, the polymerization reaction was carried out under agitation at 20° C. for 42 hours to obtain a polyamide acid solution having a solid content of 10 wt %. The results of GPC measurement of this solution revealed that the number average molecular weight (Mn) was 57,881 and the weight average molecular weight (Mw) was 147,339, with Mw/Mn being at 2.55.

Subsequently, this solution was dropped in 3.5 times by volume of methanol of 70 ml and further agitated for 1 hour, whereupon a gelled matter precipitated. The supernatant liquid was separated by decantation and 100 ml of methanol was added to the residual gelled matter and agitated, whereupon a gum-like matter precipitated. Moreover, filtration, drying and pulverization were carried out to obtain 2.0 g (yield 96%) of a yellow powder of PMDA-DDE polyamic acid.

Subsequently, 31.3 g of NMP was added to this yellow powder to prepare a 6 wt % solution, to which 9.75 g (96 mmols) of acetic anhydride and 4.50 g (57 mmols) of pyridine were added and agitated at 45° C. for 30 minutes, resulting in an agar-like matter. Further agitation at 100° C. for 2 hours lead to a gelled matter. After returning to room temperature, the reaction solution was dropped in 160 ml of methanol and further agitated for 1 hour, whereupon a yellow powder precipitated. After filtration of this yellow powder, washing with methanol was repeated, followed by drying under reduced pressure at 80° C. for 3 hours to obtain 1.59 g (yield: 83%) of a yellow powder of PMDA-DDE polyimide.

m.p.: >300° C.

The solubilities of the polyimides of BBDA-individual diamines obtained in the above Examples 6 to 9 and the PMDA-DDE polyimide obtained in Comparative Example 1 in organic solvents were evaluated according to the following procedure. The results are shown in Table 1.

(Measuring method)

5 mg of the respective polyimides was added to 1.5 g of an organic solvent and agitated at given temperatures to confirm the solubility thereof.

TABLE 1

| | BBDA polyimide | | | | | PMDA Polyimide |
|---|---|---|---|---|---|---|
| | Diamine | DDE | DA4P | DA5MG | PDA | DDE |
| Organic Solvent | Example | 6 | 7 | 8 | 9 | Comparative Example 1 |
| Dimethylsulfoxide (DMSO) | | ++ | ++ | ++ | ++ | − |
| N,N-dimethylacetamide (DMAc) | | ++ | ++ | ++ | ++ | − |
| N,N-dimethylformamide (DMF) | | +− | ++ | ++ | ++ | − |
| m-Cresol | | +− | ++ | ++ | ++ | − |
| Pyridine | | ++ | ++ | ++ | ++ | − |
| γ-Butyrolactone | | ++ | ++ | ++ | ++ | − |
| 1,4-Dioxane | | ++ | ++ | ++ | + | − |
| Tetrahydrofuran (THF) | | +− | ++ | ++ | + | − |
| Chloroform | | ++ | ++ | ++ | + | − |
| 1,2-Dichloroethane (EDC) | | ++ | ++ | ++ | + | − |
| Acetone | | − | − | − | − | − |
| Acetonitrile | | − | − | − | − | − |
| Methanol | | − | − | − | − | − |

++: dissolved at 25° C.
+: partially dissolved at 60° C.
−: insoluble at 60° C.

As shown in Table 1, it will be seen that the polyimides of the invention obtained in Examples 6 to 9 are soluble polyimides capable of being dissolve in various types of solvents including low melting organic solvents. On the other hand, the PMDA-DDE polyimide has been found to be insoluble in any of the organic solvents.

### <Preparation of a Liquid Crystal Orientation Processor>

The polyimides of BBDA-individual diamines obtained in the above Examples 6 to 8 were, respectively, weighed at 0.50 g, to which 4.50 g of γ-butyrolactone (hereinafter abbreviated as γ-BL) was added, followed by dissolution by heating at 50° C. to provide a 10 wt % solution. 1.67 g of γ-BL and 1.67 g of butylo cellosolve (hereinafter abbreviated as BCS) were added to the solution to prepare a polyimide solution having a polyimide solid content of about 6.00 wt %, 75.0 wt % of γ-BL and 20 wt % of BSC.

On the other hand, with respect to the PMDA-DDE polyimide obtained in Comparative Example 1, solubility in γ-BL was poor, so that a similar preparation of solution could not be made. Thus, a liquid crystal orientation processor containing the PMDA-DDE polyimide could not be evaluated. Hence, for comparison, a PMDA-DDE polyamic acid solution was diluted with NMP and butylo cellosolve to prepare, for evaluation, a liquid crystal orientation processor having a polyamic acid solid content of 6.0 wt %, 74.0 wt % of NMP and 20.0 wt % of BSC.

The formulations of the above liquid crystal orientation processors are shown in Table 2.

TABLE 2

| | Polyimide | | | |
|---|---|---|---|---|
| | | Acid | | |
| Liquid | | component/ | | |
| crystal | | diamine | Concentration | |
| orientation | | component | of polyimide | Solvent |
| processor | Kind | (molar ratio) | (wt %) | (wt %) |
| 1 | BBDA-DDE | 1.00/0.95 | 6.0 | γ-BL (74) |
| | | | | BC (20) |
| 2 | BBDA-DA4P | 1.00/0.91 | 6.0 | γ-BL (74) |
| | | | | BC (20) |
| 3 | BBDA- | 1.00/0.91 | 6.0 | γ-BL (74) |
| | DA5MG | | | BC (20) |
| 4 | PMDA-DDE | 1.00/1.00 | 6.0 | NMP (74) |
| (for | (polyamic acid) | | | BC (20) |
| comparison) | | | | |

### <Formation of a Liquid Crystal Orientation Film and Making and Evaluation of a Liquid Crystal Display Device>

Subsequently, the respective liquid crystal orientation processors prepared above were spin coated onto a transparent electrode-attached glass substrate and dried on a hot plate of 80° C. for 5 minutes, followed by baking on a hot plate of 210° C. for 10 minutes to form a 70 nm thick film. This film face was rubbed by means of a rubbing apparatus having a roll diameter of 120 mm and using a rayon cloth under conditions of a roll rotation frequency of 1000 r.p.m., a roll progress rate of 50 mm/second, and a push-in amount of 0.3 mm to obtain a liquid crystal orientation film-attached substrate. Two liquid crystal orientation film-attached substrates were provided, and 6 μm spacers were spread over the liquid crystal orientation film of one substrate, on which a sealing agent was printed. Another substrate was so attached that the liquid crystal orientation films were facing each other and the rubbing directions intersected at right angles, followed by curing the sealing agent to make a blank cell. A liquid crystal (MLC-2003, made by Merck KGaA) was injected into the blank cell according to a reduced pressure injection method and the injection port was sealed to obtain a twisted nematic liquid crystal cell.

With respect to the respective liquid crystal cells made in this way were subjected to evaluation of a liquid crystal orientation property and also to measurement of a pre-tilt angle and voltage retention according to the following procedures.

### [1] Evaluation of Liquid Crystal Orientation Property

For the evaluation of the liquid crystal orientation property, a roller push-in amount at the time of the indicated rubbing was changed to 0.2 mm, under which rubbing was effected in such a way that the rubbing directions were reversed upon the attachment of the substrates. In the same manner as set out above, an anti-parallel cell was made, and was sandwiched between polarizers in a parallel nicol state, followed by observation of the orientation state of the liquid crystal. The evaluation was made in the following way.
◯: Good orientation shown, with no light leakage at all
Δ: Some light leakage
x: A considerable degree of light leakage, or no orientation of a liquid crystal recognized.

### [2] Measurement of Pre-Tilt Angle

The made twisted nematic liquid crystal cell was heated at 105° C. for 5 minutes, after which the pre-tilt angle was measured according to a crystal rotation method.

### [3] Measurement of Voltage Retention

The made twisted nematic liquid crystal cell was heated at 105° C. for 5 minutes and was applied with a voltage of 4 V for 60 μs at a temperature of 90° C. The voltage after 16.67 ms was measured to calculate, as a voltage retention, how much voltage could be retained. It will be noted that for the measurement of the voltage retention, the voltage retention measuring apparatus VHR-1, made by Toyo Corporation, was used.

TABLE 3

| Liquid crystal | Liquid crystal | Pre-tilt | Voltage |
|---|---|---|---|
| orientation | orientation | angle | retention |
| processor | property | (°) | (%) |
| 1 | ◯ | 0.5 | 83 |
| 2 | ◯ | 0.1 | 67 |
| 3 | ◯ | 0.3 | 77 |
| 4 | ◯ | 0.7 | 62 |
| (for comparison) | | | |

As set forth above, when using BBDA polyimides, there is the possibility of preparing solutions by use of low boiling point solvents. The liquid crystal orientation films making use of BBDA polyimide films have a good liquid crystal orientation property. It will be seen from the values of VHR that the BBDA polyimides have more excellent electric characteristics than the orientation film using PMDA.

1. A 5-alkyl-1,2,3,4-benzenetetracarboxylic-1:2,3:4-dianhydride represented by the formula [1] 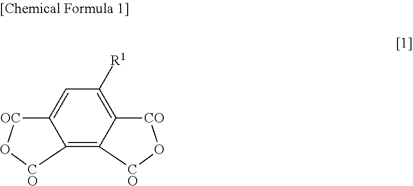
(wherein R¹ represents an alkyl group having 1 to 10 carbon atoms).

2. The 5-alkyl-1,2,3,4-benzenetetracarboxylic-1:2,3:4-dianhydride as defined in claim 1, wherein R¹ is an n-butyl group.

3. A 5-alkyl-1,2,3,4-benzenetetracarboxylic acid represented by the formula [2] 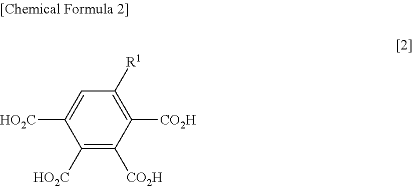
(wherein R¹ represents an alkyl group having 1 to 10 carbon atoms).

4. The 5-alkyl-1,2,3,4-benzenetetracarboxylic acid as defined in claim 3, wherein R¹ is an n-butyl group.

5. A method for producing a 5-alkyl-1,2,3,4-benzenetetra-carboxylic-1:2,3:4-dianhydride represented by the formula [1] 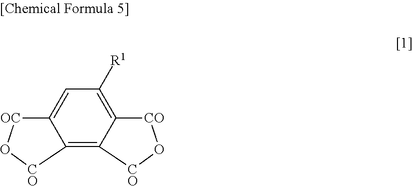
(wherein R¹ has the meaning as defined below), characterized by comprising hydrolyzing a tetraalkyl 5-alkyl-1,2,3,4-benzenetetracarboxylate represented by the formula [3] 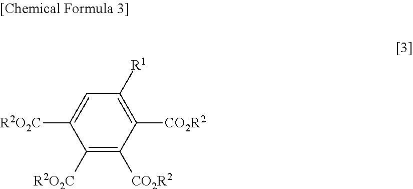
(wherein R¹ and R² independently represent an alkyl group having 1 to 10 carbon atoms) to obtain a 5-alkyl-1,2,3,4-benzenetetracarboxylic acid represented by the formula [2] 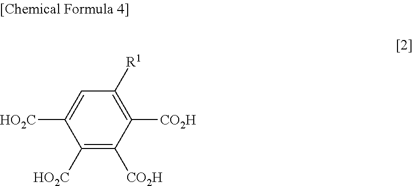
(wherein R¹ has the same meaning as defined above), and subsequently subjecting to dehydration and ring closure.

6. A method for producing a 5-alkyl-1,2,3,4-benzenetetra-carboxylic acid represented by the formula [2] 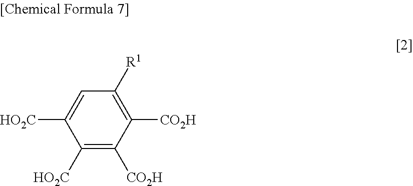
(wherein R¹ has the same meaning as defined below), characterized by comprising hydrolyzing a tetraalkyl 5-alkyl-1,2,3,4-benzenetetracarboxylate represented by the formula [3] 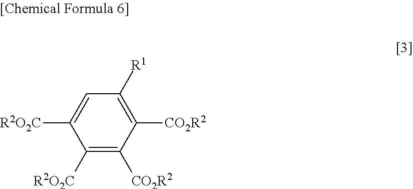
(wherein R¹ and R² independently represent an alkyl group having 1 to 10 carbon atoms).

7. A polyamic acid containing at least 10 mol % of recurring units represented by the formula [6] 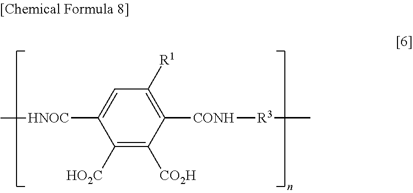
(wherein R¹ represents an alkyl group having 1 to 10 carbon atoms, R³ represents a divalent organic group, and n is an integer of 2 or over).

8. The polyamic acid as defined in claim 7, wherein R¹ is an n-butyl group.

9. A polyimide containing at least 10 mole % of recurring units represented by the formula [7] 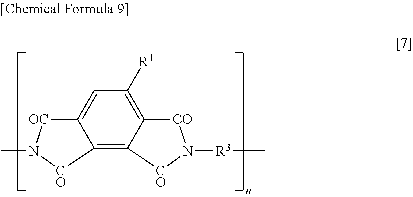
(wherein R¹ represents an alkyl group having 1 to 10 carbon atoms, R³ represents a divalent organic group, and n is an integer of 2 or over).

10. The polyimide as defined in claim 9, wherein R¹ is an n-butyl group.

11. A liquid crystal orientation processor, characterized by comprising the polyamic acid or polyimide defined in any one of claims 7 to 10.

12. A crystal liquid orientation film obtained from the liquid crystal orientation processor defined in claim 11.

13. A liquid crystal display device provided with the liquid crystal orientation film as defined in claim 12.
